(19) United States
(12) Patent Application Publication
(10) Pub. No.: US 2005/0199060 A1
(43) Pub. Date: Sep. 15, 2005

(54) Imaging tomography apparatus with a rotating part with out-of-balance compensating weights at an outer circumferential area thereof

(76) Inventors: Gunter Danz, Gross-Zimmern (DE); Hans-Jurgen Muller, Pretzfeld (DE)

Correspondence Address: SCHIFF HARDIN, LLP;PATENT DEPARTMENT, 6600 SEARS TOWER, CHICAGO, IL, 60606-6473, US

(21) Appl. No.: 11/045,894

(22) Filed: Jan. 28, 2005

(30) Foreign Application Priority Data
Jan. 28, 2004 (DE) 10 2004 004 300.0

(51) Int. Cl. G01M 1/16; A61B 5/05

(52) U.S. Cl. 73/470; 600/425; 73/462

(57) ABSTRACT

An imaging topography apparatus, in particular an x-ray topography apparatus or an ultrasonic topography apparatus, has a stationary unit with an arrangement for compensating an out-of-balance condition of an annular data acquisition device that is mounted in the stationary unit for rotation around a patient opening. Compensating weights for compensation of the out-of-balance condition are provided. For simplification of the out-of-balance condition the compensating weights are mounted at the outer circumferential area of the data acquisition device in two parallel planes that are axially separated from each other. The angle positions of the respective compensating weights are adjustable relative to each other.

### BACKGROUND OF THE INVENTION

1. Field of the Invention

The invention concerns an imaging topography apparatus, in particular an x-ray computed topography apparatus.

2. Description of the Prior Art

An x-ray computed topography apparatus is known from German OS 101 08 065. A data acquisition device or gantry, mounted such that it can be rotated around a horizontal rotational axis, is accommodated in a stationary mount. A sensor to detect an out-of-balance (unbalanced) condition of the data acquisition device is provided on the stationary mount. The sensor is connected with a device to calculate the position or positions of the rotatable data acquisition device at which a compensation weight or weights should be applied to compensate the out-of-balance condition. The balancing can ensue without the use of a specific balancing device, but a trained person is required to implement the balancing procedure, in particular for correct application of the compensation weights. The balancing procedure requires, among other things, a partial demounting of parts of the x-ray computed topography apparatus. This procedure thus is time-consuming and expensive.

U.S. Pat. No. 6,354,151 as well as German Translation 698 04 817 T2 describe an apparatus for balancing of an instrument mounting. The mass of the instrument mounting and its out-of-balance condition are thereby determined.

German Utility Model 297 09 273 discloses a balancing device for balancing rotors, Two compensation rings with a defined out-of-balance condition are provided that can be attached to one another on the rotor at suitable relative positions for compensation of an out-of-balance condition of the rotor.

German PS 199 20 699 also discloses a method for balancing rotors. Two compensation rings respectively exhibiting a defined out-of-balance condition are mounted on the rotor. To compensate the out-of-balance condition, the relative positions of the compensation rings relative to one another can be changed. For this purpose, an attachment device of the compensation rings is released, The compensation rings are held by a pawl and the rotor is rotated by a predetermined angle relative to the compensation rings. The compensation rings are subsequently locked (arrested).

To ease the locking of such compensation rings, in German OS 199 20 698 it is disclosed to fix the rings in their relative positions by means of a spring-loaded locking device on the rotor. By means of an applied force, the compensation rings can be displaced in their relative positions relative to the rotor and naturally can be locked.

To ease the identification of the correct locking position of such compensation rings, in German Utility Model 298 23 562 discloses projecting markings onto the compensation elements by means of a marking device when the rotor is located in a compensation position.

German PS 197 29 172 discloses a method for continuous compensation of an out-of-balance rotor. The out-of-balance condition of the rotor is measured by means of an out-of-balance measurement device. For compensation of the out-of-balance condition, the rotor has a number of compensation chambers filled with compensation fluid and disposed at different relative rotor positions. To compensate the out-of-balance condition, the quantity of the compensation fluid in the compensation chambers is increased or reduced in a suitable manner.

German Utility Model 299 13 630 concerns an apparatus for compensation of an out-of-balance condition in a machine tool or balancing machine. The balancing machine is thereby balanced using counterweight rotors and the position of the counterweight rotors is stored. The balancing machine is subsequently re-balanced with a component incorporated therein by displacement of the counterweight rotors. The out-of-balance condition of the component can be inferred from the deviating position of the counterweight rotors without and with the component.

German OS 197 43 577 and German OS 197 43 578 disclose a method for balancing a rotating body. Compensation masses that can be radially displaced and/or displaced in terms of their relative positions with respect to the rotating body are attached to the rotating body. At the beginning of the method, the compensation masses are initially brought into a zero position in which the vectors generated by them mutually cancel. The out-of-balance condition of the rotating body is subsequently measured and compensated by suitable shifting of the compensation masses.

The implementation of these known methods typically requires technically trained personnel. Independently of this, some of the known methods are not suited for balancing of a measurement device of a topography apparatus.

### SUMMARY OF THE INVENTION

An object of the present invention to remedy the aforementioned disadvantages according to the prior art. In particular, an imaging topography apparatus should be provided having a rotatable measurement device that can be optimally simply balanced. The balancing procedure should be fully automatically implementable, such that trained personnel are not required.

The above object is achieved in accordance with the invention by an imaging topography apparatus having a data acquisition device rotatably mounting in a stationary part, and having out-of-balance compensating weights mounted in two parallel axial planes with their angle positions being changeable relative to one another at the outer circumferential (peripheral) area of the data device acquisition. The mounting of the compensating weights at the outer circumferential area of the data acquisition device allows especially simple and automatic adjustment of the weights for compensation, an out-of-balance condition of the date acquisition device. A comprehensive compensation of radial out-of-balance vectors is possible by the compensating weights being disposed in two parallel planes that are axially separated from one another.

The out-of-balance condition can be detected by a sensor at the stationary unit that measures vibrations transferred to the stationary unit from the data acquisition device in the out-of-balance condition.

Two compensating weights in each plane are employed according to a preferred embodiment. This allows compensation in each plane according to the so-called spread angle method. Additionally the angle position of the compensating weights relative to each other is set in an appropriate way in each of the planes.

The compensating weights of each plane are guided in a track such as a groove or a similar structure. A detent for fixation of the position of each compensating weight is provided. In lieu of a detent, for example it is also possible to affix the compensating weights in their positions using magnets.

A movable barrier in the rotational path of the compensating weights on the stationary unit is provided a further embodiment. The detent can operate opposite a tangential force on each compensating weight, for instance the compensating weights can be relocated by a releasable force effected (applied) by the barrier. This allows an adjustment of the compensating weights by overcoming the opposite force of the detent. In the case of the use of magnets for the holding of compensating weights the effect of the magnetic force can be overcome by a tangential force applied to compensating weights. It is also possible, however, for instance to generate an opposing magnetic field by means of an electromagnet and therewith to release the magnetically held compensating weights to allow movement thereof.

A further sensor for determination of the rotational angle of the data acquisition device is provided a further embodiment. This allows an exact determination of the angle position of the data acquisition device or the position of the compensating weights on the data acquisition device as well as an automatic movement thereof in a new position.

A control unit can be provided for achieving such automatic adjustment, for instance a conventional controller with a microprocessor. The control unit can be connected to the sensor for measurement of the out-of-balance condition as well as the further sensor (if present) for determination of the rotation angle. Control signals for rotation of the data acquisition device for a given angle value as well as the retraction and deployment of the barrier in the rotation path of the compensating weights can be generated by the control unit. The rotation of the data acquisition device and the movement of the barrier can be controlled according to an algorithm so that the out-of-balance condition of the data acquisition device is compensated. A fully automatic compensation of the data acquisition device is thereby possible. Specially trained personnel are not necessary for the balancing procedure.

### DESCRIPTION OF THE DRAWINGS

FIG. 1 is a schematic side view of an x-ray topography device.

FIG. 2 shows the data acquisition device of the topography apparatus of FIG. 1 with compensating weights and a barrier.

FIG. 3 shows a further side view corresponding to FIG. 2.

### DESCRIPTION OF THE PREFERRED EMBODIMENTS

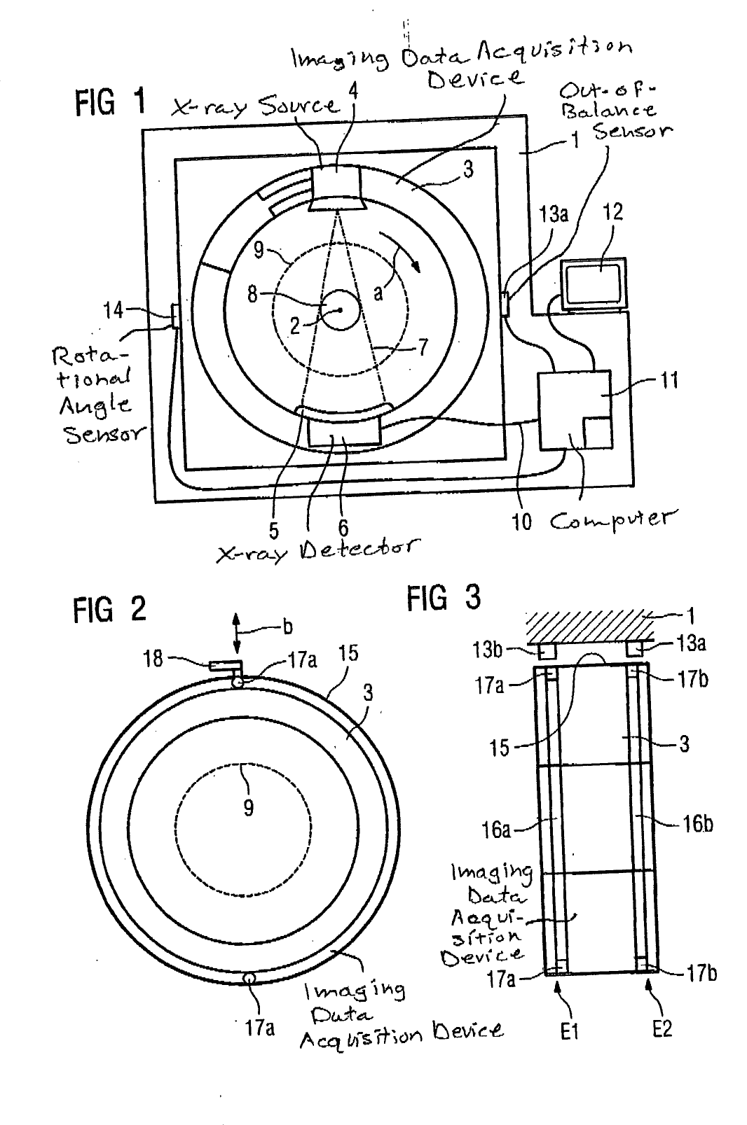

FIG. 1 schematically shows a side view of an x-ray topography apparatus with a stationary unit 1. An annular imaging data acquisition device 3 (gantry) is accommodated on the stationary unit 1 such that it can rotate around a rotation axis 2 disposed at a right angle to the plane of the drawing. The rotation direction of the imaging data acquisition device 3 is designated with the arrow a. An x-ray source 4 and an x-ray detector 5 with downstream evaluation electronic 6 are mounted on the imaging data acquisition device 3 opposite to each other, A beam fan 7 radiated by the x-ray source 4 defines a circular measurement field 8 given a rotation of the imaging data acquisition device 3. The measurement field 8 is located within a patient opening 9 indicated with the dashed line. The evaluation electronic 6 is connected with a computer 11 via a slip ring contact 10 (indicated schematically). The computer 11 has a monitor 12 for display of data. A sensor 13 for measurement of vibrations transferred to the stationary unit 1 is provided on the stationary unit 1. This is a conventional sensor with which vibrations caused by an out-of-balance condition of the imaging data acquisition device 3 and transferred to the stationary unit 1 can be measured in the radial direction and the axial direction. A further sensor 14 attached to the stationary unit 1 serves for the detection of the rotational angle of the imaging data acquisition device 3 relative to the stationary unit 1. The sensor 13 and the further sensor 14 are likewise connected with the computer 11 for evaluation of the signals measured therewith. In FIG. 1, for clarity compensation weights provided on the data acquisition device 3 are not shown.

FIGS. 2 and 3 depict schematic side views of the data acquisition device 3, wherein for clarity the x-ray source 4 and the x-ray detector 5 with the evaluation electronics 6 are not shown. Tracks such as grooves 16a and 16b are provided on an outer circumferential area 15, in a first plane E1 and in a parallel, axially separated plane E2. In every circumferential groove 16a, 16b, two movable compensating weights 17a and 17b are retained.

The compensating weights 17a, 17b are mounted so as to be movable in the respective grooves 16a, 16b. A spring loaded detent can be provided, for instance for mounting. The spring force of the detent can be overcome by the application of a tangential force and consequently the compensating weights 17a, 17b can be moved. The compensating weights 17a, 17b can, be mounted in other ways, for example frictionally or by means of magnetic force. For movement of compensating weights 17a, 17b each of the planes E1, E2 has a pawl 18 associated therewith. The pawl 18 can be moved into and out of the rotational path of the compensating weights 17a, 17b according to the arrow b.

First sensors 13a, 13b are respectively mounted on the stationary unit for each of the planes E1 and E2. The first sensors 13a, 13b register the vibrations transferred to the stationary unit in each of the planes E1, E2. By means of an appropriate evaluation program the out-of-balance vectors that produce an out-of-balance condition of the data acquisition device 3 can be determined.

The functioning of the topography device is as follows:

Initially the compensating weights 17a, 17b are disposed in a null position in each plane E1, E2, in which their vectors cancel each other. The compensating weight 17a in the first plane E1 is at the same circumferential position as the compensating weight 17b in the second plane E2.

The data acquisition device 3 is rotated. By means of the first sensors 13a, 13b the vibrations transferred to the stationary unit 1 in the plane E1 and E2 due to an out-of-balance condition of the first data acquisition device 3 are measured. Simultaneously the rotary angle of the measuring device 3 relative to the stationary unit 1 is registered by the second sensor 14. Using an appropriate calculation program stored in the computer 11 appropriate positions or angles for the compensating weights 17a, 17b are calculated for both planes E1, E2 for compensation of the out-of-balance condition of the data acquisition device 3.

For compensation, the barriers or pawls 18 are moved into the rotation paths of the compensating weights 17a, 17b. Subsequently the data acquisition device 3 is rotated according to the angle values obtained by the calculation program. The compensating weights 17a and 17b thereby are moved to the respective angle values. As soon as each compensating weight 17a, 17b has been moved to its given angle value, the corresponding pawl 18 is moved out of the rotation path of that compensating weight 17a, 17b. If present, other compensating weights located in respective planes E1, E2 are moved in the same manner. This procedure is repeated until all compensating weights 17a, 17b are located in positions obtained by the calculation program.

The method can be executed automatically. Specially trained personnel are not necessary for the balancing procedure.

Although modifications and changes may be suggested by those skilled in the art, it is the intention of the inventors to embody within the patent warranted hereon all changes and modifications as reasonably and properly come within the scope of their contribution to the art.

1. An imaging topography apparatus comprising:
a stationary unit having a patient opening therein;
an imaging data acquisition device rotatably mounted in said stationary unit for rotation around said patient opening said imaging data acquisition device having an outer circumferential area; and
in each of two planes in said imaging data acquisition device, axially separated from each other, a plurality of compensating weights for compensating an out-of-balance condition of said imaging data acquisition device, said compensating weights in each plane being movably mounted in said outer circumferential area along a rotational path defined by rotation of said imaging data acquisition device allowing selective changing of an angle between any of the compensating weights in the plane, said angle having a vertex coinciding with a rotational axis of said imaging data acquisition device.

2. An imaging topography apparatus as claimed in claim 1 wherein said imaging data acquisition device is an x-ray topography device.

3. An imaging topography apparatus as claimed in claim 1 wherein said imaging data acquisition device is an ultrasound topography device.

4. An imaging topography apparatus as claimed in claim 1 wherein, in each of said planes, said imaging data acquisition device has a track in which the compensating weights in that plane are guided.

5. An imaging topography apparatus as claimed in claim 1 wherein each of said compensating weights has a detent for fixing a position of that compensating weight at said outer circumferential area.

6. An imaging topography apparatus as claimed in claim 1 comprising, in each of said planes, a movable barrier movable into and out of said rotational path of the compensating weights in that plane for setting respective positions of the compensating weights in that plane as said imaging data acquisition device rotates.

7. An imaging topography apparatus as claimed in claim 6 wherein each of said compensating weights has a detent that applies a force to that compensating weight for fixing a position of that compensating weight at said outer circumferential area, and wherein said movable barrier applies a force to each compensating weight that overcomes the force applied by said detent.

8. An imaging topography apparatus as claimed in claim 6 comprising a control unit connected to and operating the respective movable barriers in said planes for automatically setting the respective positions of said compensating weights in each of said planes.

9. An imaging topography apparatus as claimed in claim 8 comprising a sensor at said stationary unit and connected to said control unit, said sensor generating a signal, supplied to said control unit, indicating occurrence of said out-of-balance condition.

10. An imaging topography apparatus as claimed in claim 8 comprising a further sensor at said stationary unit, connected to said control unit, that generates a signal, supplied to said control unit, indicating a rotational angle of said imaging data acquisition device.

11. An imaging topography apparatus as claimed in claim 10 wherein said control unit employs the respective signals from said sensor and said further sensor in an algorithm for setting the respective positions in said planes of said compensating weights.
